(19) United States
(12) Patent Application Publication
(10) Pub. No.: US 2004/0146876 A1
(43) Pub. Date: Jul. 29, 2004

(54) Complex element micro-array and methods of use

(76) Inventor: Peter Estibeiro, Midlothian (GB)

Correspondence Address: Fleshner & Kim, P O Box 221200, Chantilly, VA, 22153-1200, US

(21) Appl. No.: 10/469,949

(22) PCT Filed: Mar. 7, 2002
(86) PCT No.: PCT/GB02/01021

(30) Foreign Application Priority Data
Mar. 8, 2001 (GB) 0105787.6

(51) Int. Cl. C12Q 1/68; C12M 1/34

(52) U.S. Cl. 435/6; 435/287.2

(57) ABSTRACT

A device and method for mapping mRNA transcripts and determining regions that may be effective targets for antisense mediated gene knockdown. Multiple oligonucleotides are immobilised at the same position on an array in the form of complex elements. The mixture of oligonucleotides comprising each complex element is such that data can be obtained and interpreted when labelled RNA is added to the array.

This invention relates to a device and method for mapping mRNA transcripts and determining regions that may be effective targets for antisense mediated gene knockdown. In particular, the method is based on multiple oligonucleotides being immobilised at the same position on an array in the form of complex elements, such that the total number of complex elements on the array is between 4,000 and 250,000. The mixture of oligonucleotides comprising each complex element is such that data can be obtained and interpreted, when labelled RNA is added to the array, from the equivalent of between 1×10⁶ and 2×10⁹ individual six to fifteen base oligonucleotides.

Antisense as a means of controlling gene expression for research or therapeutic purposes was first described in the 1970s. Since then, much effort has been put into understanding how antisense works and into developing methods to map effective antisense targets.

Antisense works by introducing a short synthetic nucleic acid, the antisense agent, that is complimentary to a target mRNA, into a cell. The antisense agent binds to its target mRNA and prevents translation by mechanisms thought to involve both tagging for degradation by endogenous nucleases and a physical hindrance of translocation or translation.

The design of antisense agents is complicated by the fact the mRNA has extremely complex secondary and tertiary structures. At least 90% of the nucleotide sequence of any given mRNA is involved in intra-molecular interactions within the secondary and tertiary structure of the molecule, and is thus unavailable to participate in inter-molecular interaction with an antisense agent. The key to the design of a successful antisense agent is to identify the limited regions of a potential mRNA target that are available for inter-molecular hybridisation. Antisense agents targeted specifically to these accessible regions have a high probability of binding to the target mRNA in vivo, and effectively knocking down the level of expression of its encoded product.

There are a number of in vitro experimental methods available in the prior art that can be used to effectively target antisense agents. Generally these experimental methods rely on using libraries of oligonucleotides to mediate cleavage of in vitro RNA transcripts by RNaseH, or to bind to the RNA and cause retardation of a labelled species during gel electrophoresis, or to bind to the RNA on a array of separate elements, such that the signal from each element can be detected and correlated with ability to bind in that position.

Successful methods depend on knowing the sequence of the target mRNA and designing a library of overlapping oligonucleotides generally of up to twenty-five nucleotides in length. The sequence of the target mRNA is represented in the oligonucleotide library, such that the first oligonucleotide may be complimentary to positions one to fifteen on the target mRNA, the second will be complimentary to two to sixteen, and the third three to seventeen, etc.

Methods have been proposed in the prior art, whereby antisense agents can be designed using a device that is not dependent on advanced knowledge of the target sequence. Such a device is proposed to comprise an array of oligonucleotides immobilised on a glass or plastic support, such that all possible sequence combinations are represented by oligonucleotides of between four and eight nucleotide units. This type of device is disclosed in International Patent No WO98/15651 and also in U.S. Pat. No. 6,054,270. In this patent and patent application each oligonucleotide is proposed to be physically separate on the array and after hybridisation of the target mRNA and washing off of unbound material, the signal is detected from the bound oligonucleotides. By knowing which sequences are present, and at which physically separate location on the array, the sequence of target mRNA that is accessible to inter-molecular hybridisation can be inferred. The inferred sequence is likely to be an effective target for antisense mediated gene knockdown.

Both WO98/15651 and U.S. Pat. No. 6,054,270 describe arrays comprising all possible four to eight base sequence combinations. While this is technologically feasible, with four base sequence combinations requiring 256 elements on an array to represent all sequence combinations, and eight base sequence combinations requiring 65,536 elements on an array to represent all sequence combinations, there are difficulties with using such short four to eight base oligonucleotides as the basis for an array. The difficulty with using short four to eight base oligonucleotides as the basis for an array to map RNA structure and define regions that are effective antisense targets, is that the interaction between the oligonucleotides on the array and the labelled transcript applied to it under hybridising conditions is very weak. Under normal washing conditions, the transcript is washed off and no signal is detected. Increasing salt concentration or decreasing temperature tends to increase non-specific background, but does not improve the signal. In Patent Application WO98/15651 it is demonstrated that a signal can be detected by hybridising RNA to four base oligonucleotides. However, under the conditions in the described protocol, there is a requirement that the RNA rather than the oligonucleotides is immobilised to the solid support and that the oligonucleotides are applied in solution to denatured RNA. Under these conditions, it is unlikely that the RNA would be folded into an authentic representation of its in vivo structure, and the method demonstrated would not map the structure of the RNA in a suitable manner to target antisense.

It can therefore be seen that there is a requirement for an array which can contain all possible combinations of oligonucleotides of length greater than ten nucleotides. If each oligonucleotide was separately attached to the array this would be an unfeasibly large number. It would also be useful if the RNA could be added to the array in manner that allows it to retain its natural structure.

A first object of the present invention is to provide a device for mapping native RNA transcripts and determining regions that may be effective targets for antisense mediated gene knockdown.

A second object of the present invention is to provide a method of representing all possible combinations of a specific length or lengths of oligonucleotides on an array or micro-array.

A third object of the present invention is to provide a method of assigning oligonucleotide sequences to particular elements on an array.

A yet further object of the present invention is to provide a method for meaningful interpretation of arrays of complex elements to allow mapping of RNA structure and design of antisense agents.

According to a first aspect of the present invention, there is provided a device that comprises all possible oligonucleotides of a defined length or lengths on an array, wherein at least one element on the array is a complex element comprising multiple oligonucleotides of defined sequence immobilised to a support with the sequences of elements at every position on the array being known.

The oligonucleotides can be made of any natural or synthetic or modified nucleotide, or deoxynucleotide.

Optionally the support is made of glass.

Alternatively, the support is made of plastic.

Alternatively the support may be made of any appropriate material.

Preferably the oligonucleotides are not physically separated within each complex element.

Preferably all possible six to nine or ten to fifteen base oligonucleotides are represented on the array.

Preferably the array is a micro-array.

Preferably the number of oligonucleotides that make up a complex element are between 2 and 10,000.

Preferably the array will comprise between 96 and 1 million physically separate complex elements.

Preferably the complex elements are immobilised to a support using standard linking methods.

Optionally the complex elements are immobilised to a support using amino linkers.

A further option is that the complex elements are immobilised to a support using biotin/streptavidin interactions.

Preferably the oligonucleotides are immobilised at their 5′ end.

Alternatively, the oligonucleotides may be immobilised at their 3′ end.

Optionally any appropriate method of immobilising the oligonucleotides to the array may be used.

Preferably the oligonucleotides are spaced away from the solid support.

Most preferably the oligonucleotides are spaced away from the solid support using a chemical spacer of between six and forty carbon atom equivalents.

Most preferably the chemical spacer is linked between an anchor group on the array and the beginning of the oligonucleotide sequence.

Optionally the specific sequence of the oligonucleotides may be spaced away from the array by extending the 5′ or 3′ ends of the oligonucleotide using a plurality of spacing nucleotides or nucleotide analogues.

For example the six base sequence 5CGGAAC3′ may be spaced from the array by making it 5′AAAAAAAAAAACGGAAC3′ or 5′CGGACAAAAAAAAAAA3′.

Optionally the spacing nucleotides can be any natural or synthetic nucleotide or nucleotide analogue and can be a homopolymer or a heteropolymer.

Nucleotide in this document is also taken to mean deoxynucleotide or any modified nucleotide or deoxynucleotide.

Optionally one method of spacing may be used in conjunction with another method of spacing.

According to a second aspect of the present invention, there is also provided a method of producing complex elements for attachment to the device of the first aspect comprising mixing together a specified number of pre-determined length and sequence oligonucleotides at specified concentrations.

Preferably equal amounts of individual oligonucleotides are mixed together.

Optionally unequal amounts of individual oligonucleotides may be mixed together.

Preferably the individual oligonucleotides which will make up one complex element are selected such that they will not readily hybridise to each other.

Preferably each oligonucleotide within each complex element is selected such that it will have less than 60% complimentarily to any other oligonucleotide in the complex element.

Preferably each oligonucleotide within each complex element is selected so that it will have five or less bases of contiguous complimentarity with any other oligonucleotide in the complex element.

Preferably computer based algorithms are used to assign sequences to complex element groups.

According to a third aspect of the present invention, there is provided a method of interpreting complex element arrays, as described in the previous aspects, and mapping accessible regions of applied native RNA by identifying the binding of applied labelled RNA to oligonucleotides in the complex elements present on an array, wherein the array is:

a) scanned for signals caused by hybridisation between the labelled RNA applied to the array and any of the oligonucleotides that make up a complex element; and

b) said signals are compared against the known sequences in the originating complex element; and

c) overlaps between elements are identified.

Preferably the RNA is labelled by any standard means

Optionally the RNA is fluorescently labelled.

Alternatively, the RNA is radiolabelled.

Alternatively the RNA is unlabelled and interaction with the complex elements is by extension of interacting oligonucleotides using an RNA dependent enzymatic activity to incorporate label onto free 3′ ends of those oligonucleotides that are able to base-pair with the applied RNA.

Preferably the RNA dependent enzymatic activity is reverse transcriptase activity, examples of suitable enzymes are AMV reverse transcriptase or M-MuLV reverse transcriptase. Engineered reverse transcriptase lacking RNaseH activity can also be used, an example of such is Expand reverse transcriptase available commercially from Roche.

Preferably the signals are detected by fluorescence, phosphorimaging or autoradiography.

This invention is not limited by the means of detection.

Preferably comparison of the sequences within each complex element from which a signal is obtained will give an overlapping pattern that infers a contiguous accessible sequence.

Preferably provision is made for scanning complex elements in which the target sequence occurs with a single or multiple mis-match.

Preferably the comparison of a signal from the primary complex element with a signal from mis-matched complex elements will give an indication of the kinetics of binding.

Preferably the amplitude of the signal will be examined, as this will be higher when binding occurs to a number of non-overlapping sequences within the same complex element.

Optionally the signal from a labelled transcript, which is bound to a single or small number of oligonucleotides, (comprising less than 20% of a complex element mixture) can be amplified.

Most preferably the amplification will be by indirect labelling of the transcript by two-stage antibody binding.

Preferably, in all of the abovementioned aspects, the RNA that is transcribed to be applied to an array is transcribed in vitro from a full length or partial cDNA clone under non-denaturing conditions.

Most preferably the nascent RNA is maintained at all times under non-denaturing conditions.

In order to provide a better understanding of the present invention, embodiments of the invention will now be described by way of example only, and with reference to the following Figures, in which:

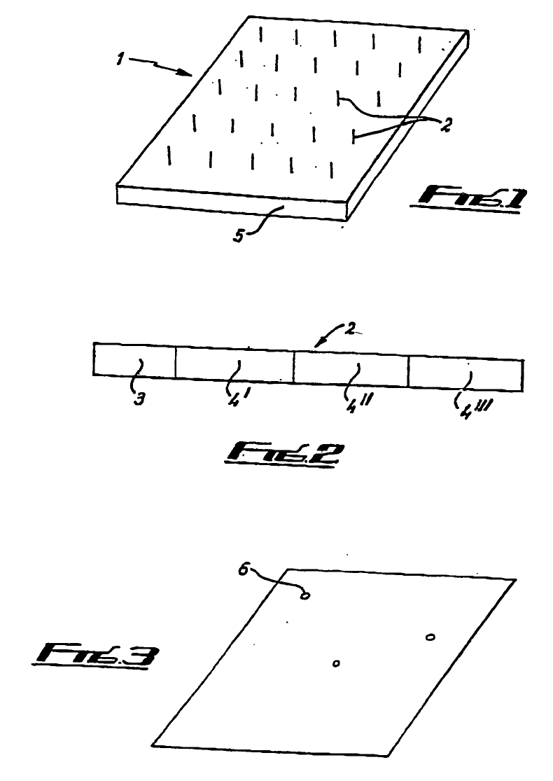

FIG. 1 is a perspective view of an array according to this invention;

FIG. 2 is a diagram of a complex element according to this invention; and

FIG. 3 is a sketch drawing of the results which may arise when using an array according to this invention.

The invention is aimed at the problem of identifying accessible sites within a native RNA in vitro transcript that can be used to target antisense research tools and therapeutics against a corresponding mRNA or other in vivo transcript. The method addresses the requirement for a tool that will map accessible regions on any native RNA. A key is that it can be a non-molecule specific antisense design tool that uses combinatorial libraries of oligonucleotides 4 of between six to fifteen bases in length, but most likely ten to fifteen bases, immobilised on a support of glass or plastic or other appropriate material 5. The support 5 may be a typical array or micro-array support. Each complex element 2 comprises N individual oligonucleotides 4 which are not physically separated, where N is between 2 and 10,000. The array 1 itself will comprise between 96 and 1 million separate complex elements 2.

In the first aspect of this invention, a multiplexed array 1, as shown in FIG. 1, is provided that comprises all possible six to nine and ten to fifteen base oligonucleotides 4. Each complex element 2 on the array 1 comprises multiple oligonucleotides 4 of defined sequence which are immobilised to a solid support 5, but are not physically separated within each element 4. The entire array 1 comprises a number of complex elements 2 which together represent all possible combinations of oligonucleotide sequences 4 of the specified lengths.

In a second aspect of this invention, complex elements 2, as shown in FIG. 2, are produced for use with the first aspect. To make each complex element 2, N individual oligonucleotides 4 of pre-determined length and sequence are mixed together in equal amounts (though it is possible for unequal amounts of oligonucleotide 4 to be used in the mixture to compensate for different predicted hybridisation kinetics). The complex elements 2 are then applied to the support 5, where they will be immobilised by standard methods, such as amino linkers or biotin/streptavidin interactions or any other appropriate method. Oligonucleotides 4 may be immobilised to either their 5′ or 3′ ends.

With short oligonucleotides 4 such as is proposed, spacing the oligonucleotide 4 away from the solid support 5 can increase the strength of the signal 6 which occurs when labelled RNA is detected. Therefore, individual oligonucleotides 4 are spaced away from the support 5 by a chemical spacer 3 of between six and forty carbon atom equivalents linked between the anchor group on the support 5 and the beginning of the oligonucleotide 4 sequence.

According to the third aspect of this invention, individual oligonucleotides within each complex element 2 are selected such that they will not readily hybridise to each other and are not so similar that they will give ambiguous results.

Within each complex element 2, oligonucleotides 4 are arranged so that they have less than 60% complimentarity to each other with five or less bases of contiguous complimentarily.

According to a fourth aspect of the present invention, it is possible to interpret complex element arrays 1 and map accessible regions of applied native RNAs. A signal 6 from a complex element 2 can be caused by hybridisation between labelled RNA applied to an array 1 and any of the N oligonucleotides 4 that make up that complex element 2. The key to identifying accessible regions of the mRNA is therefore to determine which of the oligonucleotides in the complex element 2 is binding to the applied labelled RNA.

A copy of the target RNA is transcribed in vitro from a full length or partial cDNA clone under conditions in which the nascent RNA can fold in a manner which is an authentic representation of its in vivo structure. The target RNA is labelled by incorporation of labelled nucleotides during transcription. Once synthesised, the target RNA is maintained under conditions that will maintain its secondary and tertiary structure.

The target RNA is then added to the array 1 and allowed to anneal to any complementary oligonucleotides 4. Any unbound RNA is then washed off. Therefore, in order to interpret the complex element array 1, the array 1 is scanned for signals, as in FIG. 3, the signals are then compared against known sequences in the originating complex element 2 and overlaps between the elements are identified. For example, an accessible region within the mRNA that is complimentary to the sequence CGGAATGCTGCCAAGGCTTCTCGAGTATG will hybridise all of the following ten base oligonucleotide sequences:

1. CGGAATGCTG
2. GGAATGCTGC
3. GAATGCTGCC
4. AATGCTGCCA
5. ATGCTGCCAA

By comparing the sequences within each complex element 2 from which a signal is obtained, an overlapping pattern will emerge and contiguous accessible sequence can be inferred. Provision is also made for scanning complex elements in which the target sequence occurs with a single or multiple mis-match. Comparing signals 6 from the primary complex element with a signal from mis-matched complex elements will give an indication of the kinetics of binding and may help to determine the likelihood that a particular accessible region on the mRNA will be a good target for antisense mediated gene knockdown.

It is also possible that two non-overlapping sequences within an extensive accessible region may occur within the same complex element. For example, CGGAATGCTG and GCTTCTCGAG will both occur in a sequence CGGAATGCTGCCAAGGCTTCTCGAGTATG. In such cases, the amplitude of the signal 6 will be higher than from a single hit complex element. In addition to the example given above, it is possible that more than one overlapping accessible region within the same mRNA may hybridise to oligonucleotides within the same complex element 2.

The strength of the signal 6 that is obtained from a complex element 2 depends to a large extent on the amount of the individual hybridising oligonucleotide 4 immobilised on the solid support 5. To get a high signal 6 the solid support 5 must have a high binding affinity for oligonucleotides 4, and this is a limitation of current binding technologies. In this case, a signal 6 can easily be detected from a hybridising oligonucleotide 4 that comprises 20% of the complex element 2 mixture. However, it is possible to amplify the signal 6 from a labelled transcript bound to the single or small number of oligonucleotides 4 comprising less than 20% of a complex element mixture 2. Such amplification will be by indirect labelling of the transcript by two stage antibody binding or similar.

To map antisense targets on mRNA, it is crucial that the target RNA is folded in such a way that it is an authentic representation of the in vivo structures. This invention therefore includes that the RNA which is transcribed to be applied to the array 1 is transcribed in vitro from a full length or partial cDNA clone under non-denaturing conditions and that the nascent RNA is maintained at all times under non-denaturing conditions.

The embodiments disclosed above are merely exemplary of the invention, which may be embodied in different forms. For example, the detection could be by RT labelling of the elements or, alternatively, detection could use FRET. Therefore, the details disclosed herein are not to be interpreted as limiting, but merely as a basis for the claims and for teaching one skilled in the art as to the various uses of the present invention in any appropriate manner.

1. A device that comprises all possible oligonucleotides of a defined length or lengths on an array, wherein at least one element on the array is a complex element comprising multiple oligonucleotides of defined sequence, immobilised to a support with the sequences of elements at every position on the array being known.

2. A device as described in claim 1, wherein the oligonucleotides can be made of any natural or synthetic or modified nucleotide or deoxynucleotide.

3. A device as described in claims 1 or 2, wherein the oligonucleotides are not physically separated within each complex element.

4. A device as described in any of the previous claims, wherein all possible six to nine or ten to fifteen base oligonucleotides are represented on the array.

5. A device as described in any of the previous claims, wherein the array is a micro-array.

6. A device as described in any of the previous claims, wherein the number of oligonucleotides that make up the complex element are between 2 and 10,000.

7. A device as described in any of the previous claims, wherein the array comprises between 96 and 1 million physically separate complex elements.

8. A device as described in any of the previous claims, wherein the complex elements are immobilised to a support using amino linker technology.

9. A device as described in any of claims 1 to 7, wherein the complex elements are immobilised to a support using biotin/streptavidin interactions.

10. A device as described in any of the previous claims, wherein the oligonucleotides are spaced away from the solid support.

11. A device as described in claim 10, wherein the oligonucleotides are spaced away from the solid support using a chemical spacer of between six and forty carbon atom equivalents.

12. A device as described in claim 11, wherein the chemical spacer is linked between an anchor group on the array and the beginning of the oligonucleotide sequence.

13. A device as described in claim 10, wherein the oligonucleotides are spaced away from the array by extending the 5′ or 3′ ends of the oligonucleotide using a plurality of spacing nucleotides or nucleotide analogues.

14. A device as described in claim 13, wherein the spacing nucleotides are any natural or synthetic nucleotide or nucleotide analogue, and can be either a homopolymer or a heteropolymer.

15. A device as described in claims 10 to 14, wherein one method of spacing may be used in conjunction with another method of spacing.

16. A method of producing complex elements for attachment to the device as described in the previous claims, comprising mixing together a specified number of pre-determined lengths and sequence oligonucleotides at a specified concentration.

17. A method as described in claim 16, wherein equal amounts of individual oligonucleotides are mixed together.

18. A method as described in claim 16, wherein unequal amounts of individual oligonucleotides are mixed together.

19. A method as described in claims 16 to 18, wherein the individual oligonucleotides which will make up one complex element are selected, such that they will not readily hybridise to each other.

20. A method as described in claims 16 to 19, wherein each oligonucleotide within each complex element is selected, such that it will have less than 60% complimentarily to any other oligonucleotide in the complex element.

21. A method as described in claims 16 to 20, wherein each oligonucleotide within each complex element is selected so that it will have five or less bases of contiguous complimentarity with any other oligonucleotide in the complex element.

22. A method as described in any of claims 16 to 21, wherein computer based algorithms are used to assign sequences to complex element groups.

23. A method of interpreting complex element arrays as described in any of the previous claims, and mapping accessible regions of applied native RNA by identifying the binding of applied labelled RNA to oligonucleotides in the complex elements present on an array, wherein the array is:
a) scanned for signals caused by hybridisation between the labelled RNA applied to the array and any of the oligonucleotides that make up a complex element; and
b) said signals are compared against the known sequences in the originating complex element; and
c) overlaps between elements are identified.

24. A method as described in claim 23, wherein the RNA is fluorescently labelled.

25. A method as described in claim 23, wherein the RNA is radiolabelled.

26. A method as described in claim 23, wherein the RNA is unlabelled and interaction with the complex elements is by extension of interacting oligonucleotides using RNA dependent enzymatic activity to incorporate label onto free 3′ ends of those oligonucleotides that are able to base-pair with the applied RNA.

27. A method as described in claim 26, wherein the RNA dependent enzymatic activity is reverse transcriptase activity.

28. A method as described in claims 23 to 27, wherein signals are detected by fluorescence, phosphorimaging or autoradiography.

29. A method as described in any of claims 23 to 28, wherein comparison of the sequences within each complex element from which a signal is obtained will give an overlapping pattern that infers a contiguous accessible sequence.

30. A method as described in any of claims 23 to 29, wherein provision is made for scanning complex elements in which the target sequence occurs with a single or multiple mis-match.

31. A method as described in claim 30, wherein the comparison of the signal from the primary complex element with a signal from the mis-matched complex elements will give an indication of the kinetics of binding.

32. A method as described in claims 23 to 31, wherein the amplitude of the signal is examined to determine whether binding has occurred to a number of non-overlapping sequences within the same complex element.

33. A method as described in claims 23 to 32, wherein the signal from a labelled transcript is amplified.

34. A method as described in claim 33, wherein the amplification will be by indirect labelling of the transcript by two-stage antibody binding.

35. A method or device as described in any of the previous claims, wherein the RNA that is transcribed to be applied to an array is transcribed in vitro from a full length or partial cDNA clone under non-denaturing conditions.

36. A method or device as described in any of the previous claims, wherein the nascent RNA is maintained at all times under non-denaturing conditions.
